SEQUENCE LISTING (9 sequences)

SEQ ID NO: 1 - protein, 337 aa, homo sapiens
  PEPTIDE (1)..(337)
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 2 - protein, 99 aa, homo sapiens [sequence omitted]

SEQ ID NO: 3 - protein, 337 aa, homo sapiens
  PEPTIDE (1)..(337)
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 4 - protein, 476 aa, homo sapiens
  PEPTIDE (1)..(476)
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 5 - protein, 476 aa, homo sapiens
  PEPTIDE (1)..(476)
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 6 - DNA, 1428 bp, homo sapiens
  gene (1)..(1428)
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggcggttctggtggtggcggtagtggcggcggaggaagcggtggtggcggttccggttcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccggttctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtgggaaaggagggagctactctaaggctgagtggagcgacagtgcccaggggtctgagtctcacagcttg

SEQ ID NO: 7 - DNA, 1428 bp, homo sapiens
  gene (1)..(1428)
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggcggttctggtggtggcggtagtggcggcggaggaagcggtggtggcggttccggttcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccggttctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtgggaaaggagggagctactctaaggctgagtggagcgacagtgcccaggggtctgagtctcacagcttg

SEQ ID NO: 8 - protein, 376 aa, herpes simplex virus
  PEPTIDE (1)..(376)
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 9 - DNA, 1128 bp, herpes simplex virus
  gene (1)..(1128)
atggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaac

(19) United States
(12) Patent Application Publication
(10) Pub. No.: US 2021/0024884 A1
(43) Pub. Date: Jan. 28, 2021

(54) SAFE IMMUNO-STEALTH CELLS

(71) Applicant: Novo Nordisk A/S, Bagsvaerd (DK)

(72) Inventors: Jay Chaplin, Seattle, WA (US); Ulrik Doehn, Oelstykke (DK)

(21) Appl. No.: 17/069,896

(22) Filed: Oct. 14, 2020

Related U.S. Application Data
(63) Continuation of application No. PCT/EP2020/067995, filed on Jun. 26, 2020.

(30) Foreign Application Priority Data
Jun. 27, 2019 (EP) 19182963.9
Dec. 19, 2019 (EP) 19218122.0
Apr. 20, 2020 (EP) 20170447.5

(51) Int. Cl. C12N 5/0735; C12N 15/87; A61K 35/12

(52) U.S. Cl. CPC C12N 5/0606; A61K 35/12; C12N 15/87

(57) ABSTRACT

The present invention relates to safe and immuno-stealth implantable cells and their use to prevent, treat or cure a disease.

### CROSS-REFERENCE TO RELATED APPLICATIONS

This application is a Continuation of International Application PCT/EP2020/067995, filed Jun. 26, 2020, which claims priority to European Patent Applications 19182963.9, filed Jun. 27, 2019, 19218122.0, filed Dec. 19, 2019, and 20170447.5, filed Apr. 20, 2020; the contents of which are incorporated herein by reference.

The present invention relates to the field of mammalian cells, and to the use of such cells as donor cells for implantations.

### SEQUENCE LISTING

The instant application contains a Sequence Listing which has been submitted in ASCII format via EFS-Web and is hereby incorporated by reference in its entirety. Said ASCII copy, created on Sep. 21, 2020, is named 190045US01_SeqList.txt and is 24 kilobytes in size.

### BACKGROUND

Universally transplantable tissues and/or cells are being researched on in hope for significant benefits such as reduction in graft rejection risk e.g. in the context of non-matching donor/recipient immunological profiles or of autoimmune conditions such as Type 1 diabetes mellitus (T1 D).

To limit the risk of draft rejection, auto-transplantation is an option whereby stem cells are extracted from a patient, expanded, differentiated and transplanted back into the same patient. However, this process is technically very difficult and expensive.

Tissue mismatch rejection is mediated by class I HLA (Human Leucocyte Antigen) peptide complexes and subsequent T-cell based tissue destruction. The depletion of class I HLA peptide complexes absolves the requirement for tissue matching for most cells.

There exist 6 class I HLA peptide complexes: highly polymorphic class I HLA peptide complexes HLA-A, HLA-B and HLA-C, and less polymorphic class I HLA peptide complexes HLA-E, -F, and -G.

Depletion of class I HLA peptide complexes can be achieved through either of two pathways:

1) By direct removal of all six highly polymorphic class I HLA alleles, or

2) By elimination of the beta 2 microglobulin (B2M) protein. B2M is necessary for the translocation of all HLA-I complexes to the cell surface. The absence of B2M protein renders the cell's surface devoid of all class I HLA peptide complexes.

While pan-class I HLA deficient cells are protected from mismatch rejection, they are susceptible to Natural Killer cell rejection (NK cells) due to the absence of class I HLA-E complexes. When present on the cell surface, class I HLA-E complexes deliver an inhibitory signal to NK cells. In absence of HLA-E complexes, the loss of this inhibitory signal results in lysis of the HLA deficient cell by NK cells.

Attempts to solve this issue of NK lysis rely on the expression of engineered variants of B2M protein fused to HLA-E protein (WO19032675). One approach (Gornalusse, et al. Nature Biotechnology 2017) is to pre-build a signal peptide (HLA class I leader peptide sequence) in the fusion protein in the form of a signal peptide/B2M/HLA-E trimer to increase stability and membrane expression of the complex. Most significant development programs use fusion constructs including a fused (or “pre-bound”) HLA-G derived signal peptide as HLA class I leader peptide.

An acknowledged issue in generating HLA-deficient cells (also named “universal donor” cells) is that they become silent to immune surveillance for viral infection or neoplastic transformation. There remains an associated risk that upon viral infection or malignant dedifferentiation, the cells are no longer subject to regular immune surveillance, and this triggers safety concerns.

There remains a need for improved safe universal donor cells.

Gornalusse G. et al (Nature Biotechnology 2017) disclose HLA-E expressing pluripotent stem cells.

WO2012145384 discloses B2M deficient cells.

U.S. Pat. No. 8,586,358B2 discloses HLA homozygous cells that are homozygous for a HLA haplotype.

US20040225112A1 discloses genes encoding single chain HLA-E proteins to prevent NK cell-mediated cytotoxicity.

Deuse et al (Nature Biotechnology, 2019) discloses knocked out B2M and CIITA and added CD47.

WO19032675 discloses an isolated genetically modified T-cell comprising sequences encoding a fusion protein comprising a B2M protein and HLA-E and/or HLA-G protein.

WO18005556 allegedly discloses cells comprising an MHC-E molecule.

Young et al. Cancer Gen. Therapy (2000), 7:240-246 discloses ganciclovir mediated cell killing using the Herpes Simplex Virus-Thymidine Kinase (HSV-TK) gene.

### SUMMARY

In one aspect the present invention provides a mammalian cell comprising a B2M/HLA-E gene, such as B2M/HLA-E*0101 and B2M/HLA-E*0103 genes, wherein said mammalian cell comprises no other expressible B2M genes. In an embodiment, said mammalian cell has knock-ins of at least 4 HSV-TK genes at distinct and known locations.

In another aspect the present invention provides a mammalian cell which has knock-ins of B2M/HLA-E genes, such as both B2M/HLA-E*0101 and B2M/HLA-E*0103 genes into an otherwise B2M and HLA-II deficient cell, for example CIITA deficient cell.

In another aspect the present invention provides a mammalian cell comprising a B2M/HLA-E gene wherein said mammalian cell comprises no other expressible B2M genes, is CIITA deficient and has knock-ins of 4 HSV-TK genes at distinct and known locations.

In another aspect the present invention provides a mammalian cell comprising B2M/HLA-E*0101 and B2M/HLA-E*0103 genes wherein said mammalian cell comprises no other expressible B2M genes, is CIITA deficient and has knock-ins of 4 HSV-TK genes at distinct and known locations.

In one aspect the present invention provides a method for making an implantable mammalian cell, comprising the steps of:
providing a mammalian cell,
knock-in of at least a B2M/HLA-E fusion gene, such as a B2M/HLA-E*0101 gene and/or a B2M/HLA-E*0103 gene, into said mammalian cell,
inactivating the native B2M genes of said mammalian cell, whereby said implantable mammalian cell is obtained.

In another aspect the present invention provides a method for making an implantable mammalian cell, comprising the steps of:
providing a mammalian cell,
knock-in of at least a B2M/HLA-E fusion gene, such as a B2M/HLA-E*0101 gene and/or a B2M/HLA-E*0103 gene, into said mammalian cell,
inactivating the native B2M genes of said mammalian cell,
differentiating said mammalian cell, whereby said implantable mammalian cell is obtained.

In one aspect, said mammalian cell is a human cell.

In a further aspect, said mammalian cell is a stem cell.

In one aspect, said mammalian cell is an embryonic stem cell. In another aspect, said mammalian cell is a pluripotent stem cell. In a yet another aspect, said mammalian cell is at a differentiated stage.

In an embodiment, the method of the present invention further comprises a step of knock-in of at least 4 HSV-TK genes at distinct and known locations.

In yet another aspect the present invention provides the use of a mammalian cell according to the invention for the prevention, treatment or cure of a chronic disease.

In one embodiment this chronic disease is selected from the group consisting of diabetes, type 1 diabetes, type 2 diabetes, dry macular degeneration, retinitis pigmentosa, neurological disease, Parkinson's disease, heart disease, chronic heart failure and chronic kidney disease.

The present invention provides improved universal donor cells. The cells of the present invention are more universal and safer for patients.

### Definitions

Allele:

The term “allele” as used herein means a variant of a given gene. For example, HLA-E 01:01 and HLA-E 01:03 are variants, also called alleles or isotypes, of the HLA-E gene.

B2M: The term “B2M” as used herein means beta2-microglobuline, i.e. β2 microglobulin. The term “B2M gene” designates the gene that encodes the B2M protein. The B2M protein is a subunit of all class I HLA proteins. The B2M protein is necessary for class I HLA proteins to translocate to the cell surface. In humans, the B2M gene is located on chromosome 15.

B2M Deficient Cell:

The term “B2M deficient cell” as used means a cell which has no functional B2M gene. Hence, the B2M gene may be entirely absent from the cell or it can be functionally defect, e.g. inactivated or damaged, such that it is not expressed or does not encode a functional B2M protein.

B2M/HLA-E Gene or Protein:

The term “B2M/HLA-E gene” as used herein is equivalent to “B2M/HLA-E fusion gene” and means a genetic fusion construct encoding a protein comprising a B2M part and a HLA-E part, which is equivalent to “B2M/HLA-E fusion protein”. As used herein, unless otherwise specified, the terms “B2M/HLA-E gene” and “B2M/HLA-E fusion protein” refer to any functional versions thereof, wherein the gene has the ability to express the corresponding fusion protein and wherein the expressed B2M/HLA-E fusion protein has the ability to translocate to the cell surface.

B2M/HLA-E*0101 Protein:

The term “B2M/HLA-E*0101 protein” as used herein means a fusion protein comprising a “B2M” part and a “HLA-E” part wherein the HLA-E part is of the 01:01 isotype, also called the 01:01 allele, i.e. a fusion comprising a B2M functional peptide and a HLA-E 01:01 functional peptide.

B2M/HLA-E*0101 Gene:

The term “B2M/HLA-E*0101 gene” as used herein means a genetic fusion construct encoding a B2M/HLA-E*0101 protein.

HLA/MHC:

The term “HLA” stands for Human Leucocyte Antigen. As used herein, HLA refers to the well-known HLA system responsible for the regulation of the immune system in mammalians. “HLA genes” encode for “HLA proteins” also called “MHC proteins”. “MHC” stands for “major histocompatibility complex”.

Functional “HLA proteins” (or “MHC proteins”) translocate to the cell-surface and induce an immune response as need be. In humans, the HLA genes are located on chromosome 6.

Class I HLAs proteins are heterodimers and comprise HLA-A, HLA-B and HLA-C proteins, which are highly polymorphic, and HLA-E, HLA-F and HLA-G proteins, which are less polymorphic. Class I HLA proteins are normally found on all nucleated cells' surface in humans.

The role of Class I HLA proteins is to present small peptides, herein called “endogenous peptides”, from inside the cell on the outer surface of the cell. In case of cell infection, the class I HLA peptides present to the outer cell surface a small peptide from the invader pathogen (e.g. a virus), which will be recognised as “non-self” (or “foreign” or “antigen”) and induce an immune response by destruction of the cells by the immune system. In absence of cell infection, the class I HLA peptides present to the outer cell surface an endogenous small peptide e.g. from HLA-E (HLA-E fragments) which will be recognised as “self” (or “self-antigen”) and will not induce an immune response.

Class II HLAs proteins are heterodimers and comprise HLA-DP, HLA-DM, HLA-DOA, HLA-DOB, HLA-DQ, and HLA-DR. Class II HLA proteins are normally found on professional antigen-presenting cells.

The role of Class II HLA proteins is to present antigens derived primarily from exogenous sources to the cell surface and initiate an antigen-specific immune response (via CD4(+) T-lymphocytes).

Cell Genotype:

A “gene A^{-/-} cell” means a cell wherein both copies of gene A are non-functional, e.g. deleted or otherwise disrupted. A “gene A^{+/-} cell” means a cell wherein one copy of gene A is functional, and the second copy is non-functional, e.g. is deleted or otherwise disrupted. A “gene A⁺ cell” means that the cell comprises only one copy of gene A and that said one copy of gene A is functional.

Cell Surface Phenotype:

As used herein the expression “cell surface phenotype of HLA-A/B/C^{-/-} cells” refers to a cell surface with no HLA-A, HLA-B and HLA-C proteins.

As used herein the expression “cell surface phenotype of HLA-E*0101+ HLA-E*0103+ cells” refers to a cell surface comprising HLA-E*0101 proteins and HLA-E*0103 proteins as expressed from one copy of each HLA-E allele.

CIITA/CIITA Deficient:

The term CIITA stands for “class II, major histocompatibility complex, transactivator”. The term CIITA as used herein designates the “CIITA gene” or the “CIITA protein”, i.e. the protein encoded by the CIITA gene. The CIITA protein is a transcription factor involved in the transcription of all class II HLA peptides. In the human genome, the CIITA protein is located on chromosome 16.

The term “CIITA deficient” as used herein means “without a functional CIITA gene”. A “CIITA deficient cell” means a cell that does not express a functional CIITA protein, for example the cell's CIITA gene has been knocked-out or otherwise inactivated or express a non-functional protein. In a CIITA deficient cell, all HLA class II proteins are ablated.

Distinct and Known Locations:

The expression “at known location(s)” as used herein means “in a targeted locus”. The expression refers to a gene modification, such as insertion, deletion or disruption, in a specific targeted locus (location) on the genome, as opposed to random gene modification in a random location in the genome. In particular, in connection with knock-in, the expression “at distinct and known location(s)” means that a gene of interest is not inserted at a random location in the genome but is inserted in a locus that has been predetermined and specifically targeted. This provides the advantage of ensuring a consistent level of expression of the inserted gene and for example to target safe-harbour loci.

The expression “at distinct locations” as used herein means “at different loci on the genome”. The expression refers for example to more than one nucleic acid sequence insertion, where said 2 or more nucleic acid sequences are not inserted on the same locus on the genome, i.e. on the one same position on the genome. Rather, said 2 or more nucleic acid sequences are inserted at different loci on the genome. For example, if inserted on the same chromosome, the 2 or more sequences are separated from each other by a number of nucleotides after insertion. The expression “distinct locations” may include the same locus located on 2 chromosomes of a pair of chromosomes.

EF1a Mini, EF1a, UbC, PGK, CMV and CAG Promoters:

EF1a promoter stands for human elongation factor 1α promoter, UbC promoter stands for human Ubiquitin C promoter, PGK promoter stands for mouse phosphoglycerate kinase 1 promoter, CMV promoter stands for cytomegalovirus immediate-early promoter, CAG (or CAGG) promoter stands for chicken β-Actin promoter coupled with CMV early enhancer. These promoters are constitutive promoters that may be used to drive ectopic gene expression.

UCO and UCOE:

UCOE stands for ubiquitous chromatin opening element. UCO elements prevent silencing of promotors. A UCO element may be placed upstream of a promoter.

Heterozygous for HLA-E:

A cell comprising at least two different alleles for the HLA-E gene, such as comprising a HLA-E*0101 gene and a HLA*0103 gene, is heterozygous for HLA-E.

HSV-TK Genes:

The term “HSV-TK” as used herein stands for Herpes simplex virus (HSV) thymidine kinase (TK) and designates a suicide switch system. The HSV-TK gene encodes a TK enzyme. To trigger suicide of HSV-TK⁺ cells, ganciclovir is provided to the HSV-TK⁺ cells or to the organism hosting such cells, the TK enzyme phosphorylates ganciclovir into a toxic compound that inhibits the DNA polymerase and triggers death of HSV-TK⁺ cells.

Knock-in and Knock-Out:

The term “knock-in” as used herein refers to the insertion of a gene into a genome. With knock-in techniques, the gene insertion is targeted, which means that the gene is inserted into a specific locus, in a location on the genome that has been predefined and is specifically targeted, as opposed to a random gene insertion with other genetic engineering methods.

The term “knock-out” as used herein refers to the deletion or inactivation by disruption of a gene from a genome. To achieve the deletion or disruption of a given gene of interest, knock-out techniques usually require a genetic modification in a specifically targeted location on the genome.

Several knock-in and knock-out techniques exist and are well defined in the art.

Mammalian Cell:

The term “mammalian cell” as used herein means a cell originating from a mammalian living organism, such as a mammalian animal cell or a human cell. The mammalian cell may be at an undifferentiated stage, for example at a pluripotent or multipotent stage, or at a differentiated stage, such as a fully mature stage, or at an intermediate stage of differentiation.

Matching HLA Type:

The term “matching HLA” or “matching HLA type” as used herein means a HLA isotype that is sufficiently similar between a donor cell and a host organism to not induce rejection of the donor cell by the immune system. In mammalians, HLA proteins are unique to individuals. The immune system of a host organism will recognize the “non-matching” HLA proteins on the outer cell surface of a donor cell (e.g. a grafted cell or cells in a grafted organ) as “non-self” (or “invader”) and induce an immune response and rejection of the donor cell. If the HLA proteins of a donor cell are of same or sufficiently similar isotype to the HLA proteins of a host organism, i.e. of matching HLA type with the host organism, the immune system will recognize the donor cells as “self” and will not induce rejection of the donor cell.

Polymorphic:

The term “polymorphic” as used herein means that there exist different isotypes of a given gene within a given cell. The polymorphism in the HLA system allows for a more effective and adaptive immune response.

Protein, Peptide:

Unless otherwise specified, the terms “protein” and “peptide” refer to a functional version thereof.

Safe Harbour:

The term “safe harbour site” or “safe harbour locus” or “safe genomic harbour site” as used herein means a location on the genome that is constantly expressed, that does not get silenced for example due to epigenetic silencing or downregulation of the transcription activity. AAVS1 and hROSA16 are safe harbour sites examples in the human genome. “AAVS1” stands for adeno-associated virus integration site 1 and is located on human chromosome 19. “hROSA26” stands for “human version of Gt(ROSA)26S” or “human version of ROSA26” and is located on human chromosome 3. CLYBL and CCR5 are other possible safe-harbour sites, “CLYBL” stands for “Citrate lyse beta-like” and is located on human chromosome 13, “CCR5” stands for “C—C chemokine receptor type 5” and is located on human chromosome 5.

Universally Implantable Cell, Transplantable Cell, Implantable Cell or Universal Donor Cell:

The terms “universally transplantable/implantable cell” or “universal cell” or “universal donor cell” or “transplantable cell” or “immune-safe cell” or “stealth cell” or “immuno-stealth cell” or “implantable cell” as used herein all designate a cell that can be transplanted into a host organism without being recognized as non-self hence without being rejected by the immune system of the host organism. The cell usually originates from a donor organism that is different from the host organism. A purpose of the present invention is to provide cells that may be safely implanted into a broad variety of patients without being rejected.

Implantable Mammalian Cell and Mammalian Cell:

In the context of the method(s) of the invention, method claims and method embodiments, the term “mammalian cell” refers to a cell prior to completion of the genetic modification(s) of the invention, the term “implantable mammalian cell” refers to a cell comprising the genetic modification(s) of the invention.

### FIGURES

FIG. 1 is an illustration of an embodiment of a B2M/HLA-E*0101 and B2M/HLA-E*0103 gene constructs and their knock-in in the B2M locus on human chromosome 15 according to the present invention. The illustrated gene constructs comprise a promoter, a nucleic acid sequence encoding a signal peptide, a B2M encoding nucleic acid sequence, a nucleic acid sequence encoding a (G4S)4 linker and a HLA-E*0101 encoding nucleic acid sequence for one of the gene constructs or a HLA-E*0103 encoding nucleic acid sequence for the other gene construct. The arrow [symbol] illustrates a promoter driving expression of the gene construct.

FIG. 2 is an illustration of an embodiment of 2 HSV-TK genes knock-in in safe harbour loci according to the present invention, such as the harbour loci AAVS1 (PPP1R12C) on chromosome 19, hROSA26 on chromosome 3, CCR5 on chromosome 5 or CLYBL on chromosome 13. The illustrated gene constructs comprise a promoter and a nucleic acid sequence encoding a HSV-TK protein. The arrow [symbol] illustrates a promoter driving expression of the gene construct.

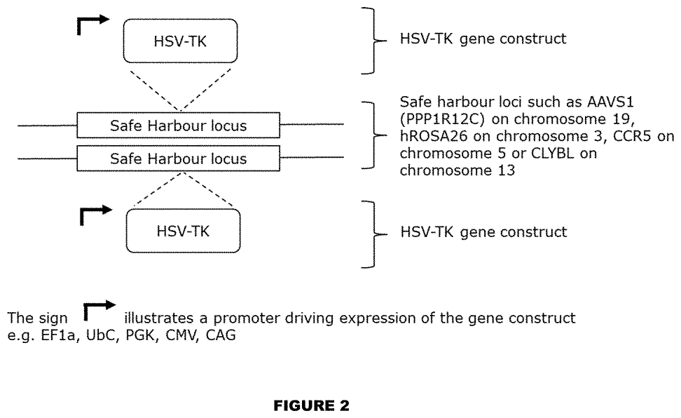

FIG. 3 shows pictures of cell cultures upon exposure to various concentrations of ganciclovir (GCV).

### DESCRIPTION

In one aspect the present invention provides a mammalian cell comprising at least one B2M/HLA-E gene wherein said mammalian cell comprises no other expressible B2M genes.

In another aspect the present invention provides a mammalian cell comprising a B2M/HLA-E gene wherein said mammalian cell comprises no other expressible B2M genes and has knock-ins of at least 4 HSV-TK genes at distinct and known locations.

In an embodiment, said mammalian cell comprises B2M/HLA-E genes. In an embodiment, said cell comprises one type of B2M/HLA-E allele, i.e. one HLA-E variant in the B2M/HLA-E fusion. In an embodiment, the HLA-E variant in the B2M/HLA-E fusion(s) is the HLA-E*01:01 allele or is the HLA-E*01:03 allele.

In an embodiment, said mammalian cell comprises two different B2M/HLA-E alleles, i.e. said cell is heterozygous for the B2M/HLA-E gene. In an embodiment, the HLA-E variants in the B2M/HLA-E fusions are the HLA-E*01:01 allele and the HLA-E*01:03 allele.

In one aspect the present invention provides a mammalian cell comprising a B2M/HLA-E*0101 or B2M/HLA-E*0103 fusion gene wherein said mammalian cell comprises no other expressible B2M genes. In one aspect the present invention provides a mammalian cell comprising B2M/HLA-E*0101 and B2M/HLA-E*0103 genes wherein said mammalian cell comprises no other expressible B2M genes.

In the present invention, the B2M/HLA-E*0101 gene encodes a B2M/HLA-E*0101 protein.

In an embodiment, the B2M/HLA-E*0101 protein comprises a B2M protein, a HLA-E*0101 protein and a linker in between the B2M protein and the HLA-E*0101 protein. In an embodiment, the B2M part is located at the N-terminus and the HLA-E part is located at the C-terminus of the B2M/HLA-E*0101 fusion protein.

In an embodiment the B2M/HLA-E*0101 protein also comprises a signal peptide.

In an embodiment, the B2M/HLA-E*0101 protein comprises a signal peptide, a B2M protein, a HLA-E*0101 protein and a linker in between the B2M protein and the HLA-E*0101 protein. In an embodiment, the signal peptide is located at the N-terminus, is followed by the B2M protein and a linker, and the HLA-E protein is located at the C-terminus of the B2M/HLA-E*0101 fusion protein.

In an embodiment, the linker between the B2M protein and the HLA-E*0101 protein is a (G4S)4 linker.

In the present invention, the B2M/HLA-E*0103 gene encodes a B2M/HLA-E*0103 protein. The term “B2M/HLA-E*0103” as used herein is intended to mean a fusion between a beta 2 microglobulin (B2M) and a HLA-E*0103.

In an embodiment, the B2M/HLA-E*0103 protein comprises a B2M protein, a HLA-E*0103 peptide and a linker in between the B2M protein and the HLA-E*0103 peptide. In an embodiment, the B2M part is located at the N-terminus and the HLA-E part is located at the C-terminus of the B2M/HLA-E*0103 fusion protein.

In an embodiment the B2M/HLA-E*0103 protein also comprises a signal peptide.

In an embodiment, the B2M/HLA-E*0103 protein comprises a signal peptide, a B2M protein, a HLA-E*0103 protein and a linker in between the B2M protein and the HLA-E*0103 protein. In an embodiment, the signal peptide is located at the N-terminus, is followed by the B2M protein and a linker, and the HLA-E protein is located at the C-terminus of the B2M/HLA-E*0103 fusion protein.

In an embodiment, the linker between the B2M protein and the HLA-E*0103 is a (G4S)4 linker.

In a preferred embodiment, the B2M/HLA-E*0101 and/or the B2M/HLA-E*0103 fusion proteins retain the ability to further bind an endogenous peptide prior to translocation to the cell surface. That is made possible by the absence of a pre-bound HLA class I leader peptide sequence (such as VMAPRTLIL) as part of said fusion protein. In an embodiment, the B2M/HLA-E*0101 and/or the B2M/HLA-E*0103 fusion proteins do not comprise a pre-bound HLA class I leader peptide sequence.

In an embodiment, the HLA-E*0101 part of the B2M/HLA-E*0101 fusion protein comprises the amino acid sequence [SEQ ID NO:01]:

GSHSLKYFHTSVSRPGRGEPRFISVGYVDDTQFVRFDNDAASPRMVPRAP
WMEQEGSEYWDRETRSARDTAQIFRVNLRTLRGYYNQSEAGSHTLQWMHG
CELGPDRRFLRGYEQFAYDGKDYLTLNEDLRSWTAVDTAAQISEQKSNDA
SEAEHQRAYLEDTCVEWLHKYLEKGKETLLHLEPPKTHVTHHPISDHEAT
LRCWALGFYPAEITLTWQQDGEGHTQDTELVETRPAGDGTFQKWAAVVVP
SGEEQRYTCHVQHEGLPEPVTLRWKPASQPTIPIVGIIAGLVLLGSVVSG
AVVAAVIWRKKSSGGKGGSYSKAEWSDSAQGSESHSL.

In an embodiment, the B2M part of the B2M/HLA-E*0101 fusion protein or of the B2M/HLA-E*0103 fusion protein comprises the amino acid sequence [SEQ ID NO:02]:

IQRTPKIQVYSRHPAENGKSNFLNCYVSGFHPSDIEVDLLKNGERIEKVE
HSDLSFSKDWSFYLLYYTEFTPTEKDEYACRVNHVTLSQPKIVKWDRDM.

In an embodiment, the HLA-E*0103 part of the B2M/HLA-E*0103 fusion protein comprises the amino acid sequence [SEQ ID NO:03]:

GSHSLKYFHTSVSRPGRGEPRFISVGYVDDTQFVRFDNDAASPRMVPRAP
WMEQEGSEYWDRETRSARDTAQIFRVNLRTLRGYYNQSEAGSHTLQWMHG
CELGPDGRFLRGYEQFAYDGKDYLTLNEDLRSWTAVDTAAQISEQKSNDA
SEAEHQRAYLEDTCVEWLHKYLEKGKETLLHLEPPKTHVTHHPISDHEAT
LRCWALGFYPAEITLTWQQDGEGHTQDTELVETRPAGDGTFQKWAAVVVP
SGEEQRYTCHVQHEGLPEPVTLRWKPASQPTIPIVGIIAGLVLLGSVVSG
AVVAAVIWRKKSSGGKGGSYSKAEWSDSAQGSESHSL.

In an embodiment, the B2M/HLA-E*0101 fusion protein comprising a (G4S)4 linker and a signal peptide comprises the amino acid sequence [SEQ ID NO:04]:

MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDMGGGGSGGGGSGGGGSGGGGSGSHSLKYFHTS
VSRPGRGEPRFISVGYVDDTQFVRFDNDAASPRMVPRAPWMEQEGSEYWD
RETRSARDTAQIFRVNLRTLRGYYNQSEAGSHTLQWMHGCELGPDRRFLR
GYEQFAYDGKDYLTLNEDLRSWTAVDTAAQISEQKSNDASEAEHQRAYLE
DTCVEWLHKYLEKGKETLLHLEPPKTHVTHHPISDHEATLRCWALGFYPA
EITLTWQQDGEGHTQDTELVETRPAGDGTFQKWAAVVVPSGEEQRYTCHV
QHEGLPEPVTLRWKPASQPTIPIVGIIAGLVLLGSVVSGAVVAAVIWRKK
SSGGKGGSYSKAEWSDSAQGSESHSL.

In an embodiment, the B2M/HLA-E*0103 fusion protein comprising a (G4S)4 linker and a signal peptide comprises the amino acid sequence [SEQ ID NO:05]:

MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDMGGGGSGGGGSGGGGSGGGGSGSHSLKYFHTS
VSRPGRGEPRFISVGYVDDTQFVRFDNDAASPRMVPRAPWMEQEGSEYWD
RETRSARDTAQIFRVNLRTLRGYYNQSEAGSHTLQWMHGCELGPDGRFLR
GYEQFAYDGKDYLTLNEDLRSWTAVDTAAQISEQKSNDASEAEHQRAYLE
DTCVEWLHKYLEKGKETLLHLEPPKTHVTHHPISDHEATLRCWALGFYPA
EITLTWQQDGEGHTQDTELVETRPAGDGTFQKWAAVVVPSGEEQRYTCHV
QHEGLPEPVTLRWKPASQPTIPIVGIIAGLVLLGSVVSGAVVAAVIWRKK
SSGGKGGSYSKAEWSDSAQGSESHSL.

In an embodiment, the B2M/HLA-E*0101 gene encoding for a B2M/HLA-E*0101 fusion protein with a (G4S)4 linker and a signal peptide comprises the nucleic acid sequence SEQ ID NO 06:

ATGTCTCGCTCCGTGGCCTTAGCTGTGCTCGCGCTACTCTCTCTTTCTGG
CCTGGAGGCTATCCAGCGTACTCCAAAGATTCAGGTTTACTCACGTCATC
CAGCAGAGAATGGAAAGTCAAATTTCCTGAATTGCTATGTGTCTGGGTTT
CATCCATCCGACATTGAAGTTGACTTACTGAAGAATGGAGAGAGAATTGA
AAAAGTGGAGCATTCAGACTTGTCTTTCAGCAAGGACTGGTCTTTCTATC
TCTTGTACTACACTGAATTCACCCCCACTGAAAAAGATGAGTATGCCTGC
CGTGTGAACCATGTGACTTTGTCACAGCCCAAGATAGTTAAGTGGGATCG
AGACATGGGTGGTGGCGGTTCTGGTGGTGGCGGTAGTGGCGGCGGAGGAA
GCGGTGGTGGCGGTTCCGGTTCCCACTCCTTGAAGTATTTCCACACTTCC
GTGTCCCGGCCCGGCCGCGGGGAGCCCCGCTTCATCTCTGTGGGCTACGT
GGACGACACCCAGTTCGTGCGCTTCGACAACGACGCCGCGAGTCCGAGGA
TGGTGCCGCGGGCGCCGTGGATGGAGCAGGAGGGGTCAGAGTATTGGGAC
CGGGAGACACGGAGCGCCAGGGACACCGCACAGATTTTCCGAGTGAACCT
GCGGACGCTGCGCGGCTACTACAATCAGAGCGAGGCCGGTTCTCACACCC
TGCAGTGGATGCATGGCTGCGAGCTGGGGCCCGACAGGCGCTTCCTCCGC
GGGTATGAACAGTTCGCCTACGACGGCAAGGATTATCTCACCCTGAATGA
GGACCTGCGCTCCTGGACCGCGGTGGACACGGCGGCTCAGATCTCCGAGC
AAAAGTCAAATGATGCCTCTGAGGCGGAGCACCAGAGAGCCTACCTGGAA
GACACATGCGTGGAGTGGCTCCACAAATACCTGGAGAAGGGGAAGGAGAC
GCTGCTTCACCTGGAGCCCCCAAAGACACACGTGACTCACCACCCCATCT
CTGACCATGAGGCCACCCTGAGGTGCTGGGCCCTGGGCTTCTACCCTGCG
GAGATCACACTGACCTGGCAGCAGGATGGGGAGGGCCATACCCAGGACAC
GGAGCTCGTGGAGACCAGGCCTGCAGGGGATGGAACCTTCCAGAAGTGGG
CAGCTGTGGTGGTGCCTTCTGGAGAGGAGCAGAGATACACGTGCCATGTG
CAGCATGAGGGGCTACCCGAGCCCGTCACCCTGAGATGGAAGCCGGCTTC
CCAGCCCACCATCCCCATCGTGGGCATCATTGCTGGCCTGGTTCTCCTTG
GATCTGTGGTCTCTGGAGCTGTGGTTGCTGCTGTGATATGGAGGAAGAAG
AGCTCAGGTGGGAAAGGAGGGAGCTACTCTAAGGCTGAGTGGAGCGACAG
TGCCCAGGGGTCTGAGTCTCACAGCTTG.

In an embodiment, the B2M/HLA-E*0103 gene encoding for a B2M/HLA-E*0103 fusion protein with a (G4S)4 linker and a signal peptide comprises the nucleic acid sequence SEQ ID NO 07:

ATGTCTCGCTCCGTGGCCTTAGCTGTGCTCGCGCTACTCTCTCTTTCTGG
CCTGGAGGCTATCCAGCGTACTCCAAAGATTCAGGTTTACTCACGTCATC
CAGCAGAGAATGGAAAGTCAAATTTCCTGAATTGCTATGTGTCTGGGTTT
CATCCATCCGACATTGAAGTTGACTTACTGAAGAATGGAGAGAGAATTGA
AAAAGTGGAGCATTCAGACTTGTCTTTCAGCAAGGACTGGTCTTTCTATC
TCTTGTACTACACTGAATTCACCCCCACTGAAAAAGATGAGTATGCCTGC
CGTGTGAACCATGTGACTTTGTCACAGCCCAAGATAGTTAAGTGGGATCG
AGACATGGGTGGTGGCGGTTCTGGTGGTGGCGGTAGTGGCGGCGGAGGAA
GCGGTGGTGGCGGTTCCGGTTCCCACTCCTTGAAGTATTTCCACACTTCC
GTGTCCCGGCCCGGCCGCGGGGAGCCCCGCTTCATCTCTGTGGGCTACGT
GGACGACACCCAGTTCGTGCGCTTCGACAACGACGCCGCGAGTCCGAGGA
TGGTGCCGCGGGCGCCGTGGATGGAGCAGGAGGGGTCAGAGTATTGGGAC
CGGGAGACACGGAGCGCCAGGGACACCGCACAGATTTTCCGAGTGAACCT
GCGGACGCTGCGCGGCTACTACAATCAGAGCGAGGCCGGTTCTCACACCC
TGCAGTGGATGCATGGCTGCGAGCTGGGGCCCGACGGGCGCTTCCTCCGC
GGGTATGAACAGTTCGCCTACGACGGCAAGGATTATCTCACCCTGAATGA
GGACCTGCGCTCCTGGACCGCGGTGGACACGGCGGCTCAGATCTCCGAGC
AAAAGTCAAATGATGCCTCTGAGGCGGAGCACCAGAGAGCCTACCTGGAA
GACACATGCGTGGAGTGGCTCCACAAATACCTGGAGAAGGGGAAGGAGAC
GCTGCTTCACCTGGAGCCCCCAAAGACACACGTGACTCACCACCCCATCT
CTGACCATGAGGCCACCCTGAGGTGCTGGGCCCTGGGCTTCTACCCTGCG
GAGATCACACTGACCTGGCAGCAGGATGGGGAGGGCCATACCCAGGACAC
GGAGCTCGTGGAGACCAGGCCTGCAGGGGATGGAACCTTCCAGAAGTGGG
CAGCTGTGGTGGTGCCTTCTGGAGAGGAGCAGAGATACACGTGCCATGTG
CAGCATGAGGGGCTACCCGAGCCCGTCACCCTGAGATGGAAGCCGGCTTC
CCAGCCCACCATCCCCATCGTGGGCATCATTGCTGGCCTGGTTCTCCTTG
GATCTGTGGTCTCTGGAGCTGTGGTTGCTGCTGTGATATGGAGGAAGAAG
AGCTCAGGTGGGAAAGGAGGGAGCTACTCTAAGGCTGAGTGGAGCGACAG
TGCCCAGGGGTCTGAGTCTCACAGCTTG.

In another aspect the present invention provides a mammalian cell which has knock-ins of both B2M/HLA-E*0101 and B2M/HLA-E*0103 genes into an otherwise B2M deficient cell.

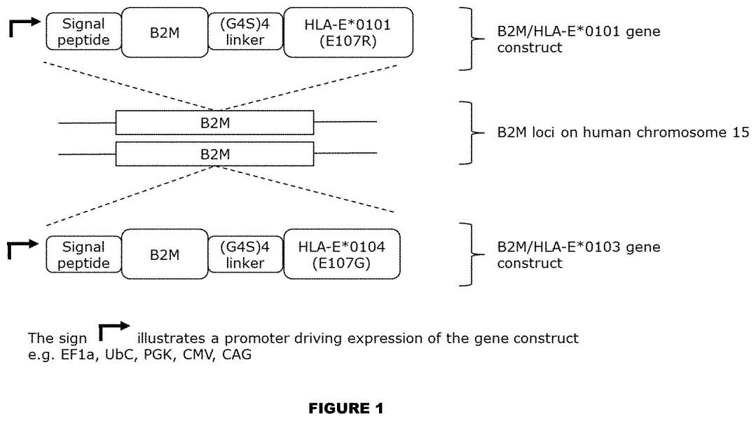

In an embodiment, the B2M/HLA-E gene is inserted at the locus of the native B2M gene, on chromosome 5 in the case of a human cell. In an embodiment, a copy of the B2M/HLA*0101 gene and a copy of the B2M/HLA*0103 gene are inserted on the locus of each of the two copies of the native B2M gene of the cell, thereby inactivating the native B2M gene. An example is illustrated in FIG. 1.

In an embodiment, the B2M/HLA gene does not comprise a sequence encoding a pre-bound HLA class I leader peptide, and the B2M/HLA protein does not comprise a pre-bound HLA class I leader peptide.

It has surprisingly been found that the use of both B2M/HLA-E*0101 and B2M/HLA-E*0103 gene fusion constructs which do not comprise a sequence encoding a pre-bound HLA class I leader peptide, into B2M-deficient cells generates the cell surface phenotype of HLA-A/B/C^{-/-} HLA-E*0101+ HLA-E*0103⁺ cells with both a high and robust HLA-E density, maximum endogenous peptide binding diversity, optimal protection against NK cell mediated non-infected target cell lysis and enhanced recognition and optimal elimination by NK cells of target mammalian cells infected with virus or other pathogen.

The present invention advantageously allows to A) constitutively increase the density of HLA-E proteins on the donor cell surface to inhibit NK cell-mediated rejection of B2M deficient cells, B) retain normal immune surveillance functions of HLA-E via native endogenous peptide binding (resulting in a slight reduction of tolerogenic capacity), C) maximize the diversity of potential endogenous peptides bound to the HLA proteins through inclusion of multiple HLA-E isotypes, and D) mitigate the risk upon viral infection or malignant dedifferentiation that the cells are no longer subject to regular immune surveillance.

To provide increased HLA-E density and achieve advantage A) through a non-native promoter, two, rather than one, alleles of the B2M/HLA-E genes are inserted in the cell. To achieve advantage B), the inventors use a B2M/HLA-E gene encoding a B2M/HLA-E fusion protein that is devoid of pre-engineered, i.e. pre-bound HLA class I leader peptide, and in turn that utilizes native endogenous peptides processing and loading mechanisms. To achieve advantage C) both two major HLA-E alleles, HLA-E*0101 and HLA-E*0103 are utilized. The two encoded HLA-E*0101 and HLA-E*0103 proteins load and present different endogenous peptide subsets, thereby increasing both the likelihood that the HLA proteins will be adequately loaded with tolerogenic endogenous peptide under normal circumstances and with activating endogenous peptide during viral infection. To achieve advantage D) the inventors have introduced 4 copies of the HSV-TK gene serving as a robust switch which can swiftly kill the cells if so desired. The combination of several modifications holds potential for both substantially better cell retention and immune surveillance under conditions of infection.

Advantageously, the combination of normal endogenous peptide loading (by not using a pre-bound peptide) and multiple HLA-E isotypes allows for expanded immune surveillance of the cells for viral and/or bacterial infection while preserving a maximally tolerogenic phenotype. During infection, several peptides from viral or bacterial pathogens can displace the normal endogenous peptides from HLA-E. When HLA-E presents pathogen-derived peptides, it stimulates NK lysis of the infected cell; contrary to HLA-E with a pre-bound peptide which would indicate a “healthy state” to NK cells, would not stimulate NK lysis and thereby provide a tolerance function). This is an important safety feature achieved with the present invention.

In an embodiment, the mammalian cell of the present invention is HLA-II deficient. In an embodiment, the mammalian cell is CIITA deficient.

Any available and relevant gene editing technology (CRISPR, TALEN, ZFN, homing endonuclease, adenoviral recombination, etc.) may be used to modify cells such that both alleles of native B2M are knocked-out while simultaneously one or more copies each of B2M/HLA-E*0101 and B2M/HLA-E*0103 genes are knocked-in.

The knock-in of B2M/HLA-E genes, such as B2M/HLA-E*0101 and B2M/HLA-E*0103 genes, may be accomplished directly over the native B2M gene locus, over other loci, such as safe harbour loci, such as the AAVS1 safe harbour locus, or any combination thereof. Any available promoter may be used for these knock-in genes, for instance a promoter selected from the group consisting of EF1a mini, EF1a, UbC, PGK, CMV and CAG. According to the present invention, the desired increase in HLA-E density is obtained via bi-allelic HLA-E knock-ins controlled by constitutively active promoters. In a native cell, endogenous HLA-E promoters are controlled by promoter INF gamma response elements.

Similarly the HSV-TK genes may be knocked-in at desired locations, i.e. at targeted loci. Any available and relevant gene editing technologies may be used.

Cells of the present invention comprise at least 4 HSV-TK genes at distinct and known locations.

In the present invention, the HSV-TK genes serve as an inducible ‘suicide switch’ system to control survival of the engineered mammalian cell for example in a host organism. The concept of a suicide switch entails genomic introduction of a gene that renders the cell sensitive to an exogenous molecule, that can be administered when needed. The HSV-TK gene encodes a thymidine kinase that converts the common small molecule antiviral drug ganciclovir into a toxic substance within the HSV-TK expressing cell. A problem with such suicide genes is that they could in theory be inactivated or eliminated by spontaneous genomic deletion or promoter slicing, resulting in the loss of the intended control by ‘suicide switch’.

In an embodiment of the invention, HSV-TK suicide genes are placed in safe harbour loci in the genome. In an embodiment of the invention, the expression of HSV-TK is driven by a promoter with an upstream UCO element. In an embodiment of the invention, the expression of HSV-TK suicide genes is driven by a UbC promoter with an upstream UCO element.

In an embodiment of the present invention, four copies of the HSV-TK suicide gene are inserted in the genome of the cell.

In an embodiment of the present invention, the knock-ins of 4 HSV-TK genes, i.e. of 4 copies of the HSV-TK gene, are at distinct locations, i.e. at locations on the genome having some separation such as to provide a safe system which is not amenable to deteriorate due to genetic rearrangements or deletions. In an embodiment the 4 HSV-TK genes are knocked-in on the same chromosome and separated from each other by at least 10 Kbp, such as at least 100 Kbp, at least 1 Mbp or at least 20 Mbp. In another embodiment the 4 HSV-TK genes are knocked-in at locations on 4 different chromosomes. In another embodiment the 4 HSV-TK genes are knocked-in at locations on 3 different chromosomes. In another embodiment the 4 HSV-TK genes are knocked-in at locations on 2 different chromosomes, such as two HSV-TK copies on same location on each both chromosomes 3 and two HSV-TK copies on same location on both chromosomes 19 in a diploid cell. In another embodiment of the present invention 2 HSV-TK genes are knock-in at safe genomic harbour sites. In another embodiment one HSV-TK gene is knocked-in to disrupt and eliminate a B2M allele. In another embodiment one HSV-TK gene is knocked-in to eliminate a CIITA allele.

Patients safety is a very important parameter in cellular therapy.

Inserting 4 copies of TK suicide gene also advantageously increases safety to patients. It was surprisingly found that a cell with 4 copies of TK suicide gene is significantly more sensitive to ganciclovir treatment than a cell with 2 copies, achieving cellular death with lower amounts of ganciclovir.

Placing TK suicide genes at known, predefined locations, advantageously increases safety to patients compared to random integration into a cell genome. Compared to random integration, targeted integration decreases the risk of disruption of important genes or of important gene expression regulation. It also decreases the risk that the suicide genes randomly integrate into a region of suboptimal expression activity. thereby ensures an optimal TK expression level.

Placing TK suicide genes at distinct locations further increases patients' safety by limiting the risk that all TK suicide gene copies get silenced or downregulated at once in the event their insertion loci get exposed to gene silencing or transcription downregulation.

Placing TK suicide genes at safe harbor loci advantageously increases safety to patients. Safe harbor loci are regions of the genome that are constantly expressed. This approach decreases the risk of the suicide genes being involuntarily silenced or downregulated, thereby increases the chance of an optimal expression level of the suicide TK protein at all time and subsequently a controlled cell death when need be upon ganciclovir administration.

It results that placing 4 TK suicide gene copies at known and distinct locations, such as safe harbor loci, provide significantly improved safety for patients receiving cell therapy as per the present invention.

In an embodiment, at least 2 HSV-TK genes are knocked-in in a safe harbour site, such as the AAVS1 gene locus or the hROSA26 gene locus or the CLYBL gene locus. In an embodiment, 2 HSV-TK genes are knocked-in in a safe harbour site, such as the AAVS1 gene locus or the hROSA26 gene locus or the CLYBL gene locus, and 2 HSV-TK genes are knocked-in in the CIITA gene locus.

In another embodiment, 2 HSV-TK genes are knocked-in in a safe harbour site, and 2 HSV-TK genes are knocked-in in another safe harbour site, and the CIITA gene locus is knocked-out. In a more specific embodiment, 2 HSV-TK genes are knocked-in in the AAVS1 gene locus, and 2 HSV-TK genes are knocked-in in the CLYBL gene locus, and the CIITA gene is knocked-out.

In an embodiment, a B2M/HLA-E gene is knocked-in in the loci of the B2M gene, thereby inactivating the cell's native B2M gene. In an embodiment, a B2M/HLA-E*01:01 gene or a B2M/HLA-E*01:03 gene is knocked-in in the loci of the B2M gene, thereby inactivating the cell's native B2M gene. In an embodiment, a B2M/HLA-E*0101 gene is knocked-in in the locus of one copy of the B2M gene, a B2M/HLA-E*0103 gene is knocked-in in the locus of the other copy of the B2M gene, thereby inactivating the cell's native B2M gene. In an embodiment, 2 HSV-TK genes are knocked-in in the loci of the AAVS1 gene and 2 HSV-TK genes are knocked-in in the loci of the CIITA gene, thereby inactivating the cell's native CIITA gene. Inactivation of the cell's native CIITA gene leads to depletion in HLA-II proteins.

In an embodiment, a B2M/HLA-E*0101 gene is knocked-in in the locus of one copy of the B2M gene, a B2M/HLA-E*0103 gene is knocked-in in the locus of the other copy of the B2M gene, 2 copies of the HSV-TK gene are knocked-in in safe harbour loci such as the AAVS1 gene, and 2 HSV-TK genes are knocked-in in the loci of the CIITA gene.

In an embodiment, a B2M/HLA-E*0101 gene is knocked-in in the locus of one copy of the B2M gene, a B2M/HLA-E*0103 gene is knocked-in in the locus of the other copy of the B2M gene, 2 copies of the HSV-TK gene are knocked-in the AAVS1 gene loci, 2 HSV-TK genes are knocked-in in the CLYBL gene loci, and the CIITA gene is knocked-out, i.e. both copies of the CIITA gene are knocked-out.

The 4 HSV-TK genes are preferably expressed to an extent where each of them alone would kill said mammalian cell upon exposure to ganciclovir.

In an embodiment, the HSV-TK protein comprises the amino acid sequence SEQ ID NO: 08:

MASYPGHQHASAFDQAARSRGHSNRRTALRPRRQQEATEVRPEQKMPTLL
RVYIDGPHGMGKTTTTQLLVALGSRDDIVYVPEPMTYWRVLGASETIANI
YTTQHRLDQGEISAGDAAVVMTSAQITMGMPYAVTDAVLAPHIGGEAGSS
HAPPPALTLIFDRHPIAALLCYPAARYLMGSMTPQAVLAFVALIPPTLPG
TNIVLGALPEDRHIDRLAKRQRPGERLDLAMLAAIRRVYGLLANTVRYLQ
CGGSWREDWGQLSGTAVPPQGAEPQSNAGPRPHIGDTLFTLFRAPELLAP
NGDLYNVFAWALDVLAKRLRSMHVFILDYDQSPAGCRDALLQLTSGMVQT
HVTTPGSIPTICDLARTFAREMGEAN

In an embodiment, the HSV-TK gene encoding a HSV-TK protein comprises the nucleic acid sequence SEQ ID NO 09:

ATGGCTTCTTACCCTGGACACCAGCATGCTTCTGCCTTTGACCAGGCTG
CCAGATCCAGGGGCCACTCCAACAGGAGAACTGCCCTAAGACCCAGAAGA
CAGCAGGAAGCCACTGAGGTGAGGCCTGAGCAGAAGATGCCAACCCTGCT
GAGGGTGTACATTGATGGACCTCATGGCATGGGCAAGACCACCACCACTC
AACTGCTGGTGGCACTGGGCTCCAGGGATGACATTGTGTATGTGCCTGAG
CCAATGACCTACTGGAGAGTGCTAGGAGCCTCTGAGACCATTGCCAACAT
CTACACCACCCAGCACAGGCTGGACCAGGGAGAAATCTCTGCTGGAGATG
CTGCTGTGGTGATGACCTCTGCCCAGATCACAATGGGAATGCCCTATGCT
GTGACTGATGCTGTTCTGGCTCCTCACATTGGAGGAGAGGCTGGCTCTTC
TCATGCCCCTCCACCTGCCCTGACCCTGATCTTTGACAGACACCCCATTG
CAGCCCTGCTGTGCTACCCAGCAGCAAGGTACCTCATGGGCTCCATGACC
CCACAGGCTGTGCTGGCTTTTGTGGCCCTGATCCCTCCAACCCTCCCTGG
CACCAACATTGTTCTGGGAGCACTGCCTGAAGACAGACACATTGACAGGC
TGGCAAAGAGGCAGAGACCTGGAGAGAGACTGGACCTGGCCATGCTGGCT
GCAATCAGAAGGGTGTATGGACTGCTGGCAAACACTGTGAGATACCTCCA
GTGTGGAGGCTCTTGGAGAGAGGACTGGGGACAGCTCTCTGGAACAGCAG
TGCCCCCTCAAGGAGCTGAGCCCCAGTCCAATGCTGGTCCAAGACCCCAC
ATTGGGGACACCCTGTTCACCCTGTTCAGAGCCCCTGAGCTGCTGGCTCC
CAATGGAGACCTGTACAATGTGTTTGCCTGGGCTCTGGATGTTCTAGCCA
AGAGGCTGAGGTCCATGCATGTGTTCATCCTGGACTATGACCAGTCCCCT
GCTGGATGCAGAGATGCTCTGCTGCAACTAACCTCTGGCATGGTGCAGAC
CCATGTGACCACCCCTGGCAGCATCCCCACCATCTGTGACCTAGCCAGAA
CCTTTGCCAGGGAGATGGGAGAGGCCAAC.

In another aspect the present invention provides a method for making an implantable mammalian cell, comprising the steps of:
providing a mammalian cell,
knock-in of at least a B2M/HLA-E fusion gene, such as a B2M/HLA-E*0101 gene and/or a B2M/HLA-E*0103 gene, into said mammalian cell,
inactivate the native B2M genes of said mammalian cell,
optionally differentiate said mammalian cell,
   whereby said implantable mammalian cell is obtained.

The order of the steps may vary where it makes sense. For example, the genetic modification steps and the cell differentiation step(s) may occur in different orders, the knock-in of a B2M/HLA-E gene may occur prior to B2M gene inactivation, the differentiation step may take place prior to B2M/HLA-E gene and/or B2M gene inactivation.

In another aspect the present invention provides a method for making an implantable mammalian cell, comprising the steps of:
providing a mammalian cell,
knock-in of at least a B2M/HLA-E fusion gene, such as a B2M/HLA-E*0101 gene and/or a B2M/HLA-E*0103 gene, into said mammalian cell,
inactivate the native B2M genes of said mammalian cell,
knock-in of at least 4 HSV-TK genes at distinct and known locations in said mammalian cell,
optionally differentiate said mammalian cell, whereby said implantable mammalian cell is obtained.

In another aspect the present invention provides a method for making an implantable mammalian cell, comprising the steps of:
providing a mammalian cell,
knock-in of at least a B2M/HLA-E fusion gene, such as a B2M/HLA-E*0101 gene and/or a B2M/HLA-E*0103 gene, into said mammalian cell,
inactivate the native B2M genes of said mammalian cell,
knock-ins of at least 4 HSV-TK genes at distinct and known locations,
inactivate the native HLA-II genes or the native CIITA genes of said mammalian cell,
optionally differentiate said mammalian cell,
   whereby said implantable mammalian cell is obtained.

In another aspect the present invention provides a method for making an implantable mammalian cell, comprising the steps of:
providing a B2M and CIITA deficient mammalian cell,
knock-in of a B2M/HLA-E fusion gene, such as a B2M/HLA-E*0101 gene and/or a B2M/HLA-E*0103 gene, into said B2M and CIITA deficient mammalian cell,
knock-ins of 4 HSV-TK genes at distinct and known locations,
   whereby said implantable mammalian cell is obtained.

In another aspect the present invention provides a method for making an implantable mammalian cell, comprising the steps of:
providing a mammalian cell,
knock-in a B2M/HLA-E*0101 gene and/or a B2M/HLA-E*0103 gene into the B2M gene of said cell,

whereby said implantable mammalian cell is obtained, is B2M deficient and expresses B2M/HLA-E*0101 and/or B2M/HLA-E*0103 proteins.

In another aspect the present invention provides a method for making an implantable mammalian cell, comprising the steps of:
providing a mammalian cell,
knock-in a B2M/HLA-E*0101 gene and/or a B2M/HLA-E*0103 into the B2M gene of said mammalian cell,
knock-ins of 4 HSV-TK genes at distinct and known locations in the genome of said mammalian cell,
optionally differentiate said mammalian cell,
   whereby said implantable mammalian cell is obtained, is B2M deficient and expresses B2M/HLA-E*0101 proteins and/or B2M/HLA-E*0103 proteins and HSV-TK proteins.

In another aspect the present invention provides a method for making an implantable mammalian cell, comprising the steps of:
a) providing a B2M deficient mammalian cell,
b) knock-in of both B2M/HLA-E*0101 and B2M/HLA-E*0103 into said B2M deficient mammalian cell,

whereby said implantable mammalian cell is obtained.

In another aspect the present invention provides a method for making an implantable mammalian cell, comprising the steps of:
a) providing a B2M and CIITA deficient mammalian cell,
b) knock-in of both B2M/HLA-E*0101 and B2M/HLA-E*0103 into said B2M and CIITA deficient mammalian cell,
c) knock-ins of 4 HSV-TK genes at distinct and known locations,

whereby said implantable mammalian cell is obtained.

It is envisioned that the mammalian cell that is subject to the genetic modifications as per the method of the invention may be at various stage of differentiation and may, as need be, be subject to further differentiation. For example, in case of a stem cell, a pluripotent cell or a cell at an early differentiation stage, this cell may be differentiated to a more advanced differentiation stage, a more mature cell type prior to implantation. The method of the invention might as well be applied to a functional cell type which does not require further differentiation prior to implantation.

In yet another embodiment the present invention provides the use of a mammalian cell according to the invention for the prevention, treatment or cure of a disease such as a chronic disease. It is envisioned that the mammalian cells and the methods of the present invention might be useful in the treatment of a wide range of chronic diseases. It is also envisioned that they might be useful in preventing chronic diseases as well as other diseases.

In an embodiment said disease is selected from the group consisting of diabetes, type 1 diabetes, type 2 diabetes, dry macular degeneration, retinitis pigmentosa, neurological disease, Parkinson's disease, heart disease, chronic heart failure and chronic kidney disease.

In an embodiment, the mammalian cell is an animal cell. In another embodiment, the mammalian cell is a human cell.

In an embodiment, the mammalian cell is an undifferentiated cell. In an embodiment, the mammalian cell is a stem cell, such as a human stem cell, a pluripotent cell, such as a pluripotent human cell or an iPS cell (induced pluripotent stem cell), such as a human iPS cell.

In an embodiment, the mammalian cell of the invention is an undifferentiated cell, such as stem cell, pluripotent cell or iPS cell, that is further differentiated into a functional cell type.

In another embodiment, the mammalian cell is a differentiated cell.

In an embodiment, the mammalian cell is a human differentiated cell derived from a stem cell, from a pluripotent cell or from an iPS cell of the invention.

In particular embodiments of the present invention, the mammalian cell is a differentiated cell selected from the below list.

Said differentiated cell may be derived from a stem cell, a pluripotential cell or an iPS cell of the invention according to one of the differentiation methods described in the publications referred to in the below list:
a beta cell, for example an INS+ and NKX6.1+ double positive cell or a C-peptide+/NKX6.1+ double positive cells, an insulin producing cell, an in vitro derived beta-like cell, a pancreatic endocrine cell or an endocrine cell, as obtainable by the method described in WO2017/144695
an endocrine progenitor cell or a NGN3+/NKX2.2+ double positive cell, as obtainable by the method described in the patent application WO2015028614
a neural cell, such as a neuron, an interneuron cell, an oligodendrocyte, an astrocyte, a dopaminergic cell, as obtainable by the methods described in Nolbrant S. et al., Nat. Protoc. 2017 September, 12(9):1962-1979; Kirkeby A. et al., Cell Rep. 2012 Jun. 28, 1(6):703-14; Aktinson-Dell R. et al, Adv Exp Med Biol. 2019, 1175:383-405; Ni P. et al, Mol Ther Methods Clin Dev. 2019 Apr. 8, 13:414-430;
an exosome cell, such as ESCs (Embryonic Stem Cell) or NSCs (Neuronal Stem Cell), or an exosome cell derived from a ESC or NSC as obtainable by the methods described in Chen B. Stem Cell Res Ther. 2019 May 21, 10(1):142; Sun X. et al, Front Cell Neurosci. 2019 Sep. 3, 13:394; Dougherty J. A. et al., Front Physiol. 2018 Dec. 14, 9:1794; Candelario K. M. et al., J Comp Neurol. 2019 Nov. 19; Yang R. et al., Front Immunol. 2019 Oct. 16, 10:2346;
an immune cell, such as a T cell, a NK cell, a macrophage, a dendritic cell as obtainable by the methods described in Ackermann M. et al., Nat Commun. 2018 Nov. 30; 9(1):5088; Good M L. et al. J Vis Exp. 2019 Oct. 24 (152); Zhu H. et al. Methods Mol Biol. 2019, 2048:107-119; Kitadani J. et al, Sci Rep. 2018 Mar. 15; 8(1):4569;
a hepatocyte as obtainable by the method described in Li Z. et al. Cell Death Dis. 2019 Oct. 10, 10(10):763;
a stellate cell as obtainable by the methods described in Coll M. Cell Stem Cell. 2018 Jul. 5, 23(1):101-113;
a fibroblast, a keratinocyte or a hair cell as obtainable by the methods described in Miyake T. Int J Radiat Oncol Biol Phys. 2019 Sep. 1, 105(1):193-205;
an inner ear cell as obtainable by the method described in Jeong M. et al, Cell Death Dis. 2018 Sep. 11; 9(9):922;
an intestinal cell or organoid cell as obtainable by the methods described in Negoro R. et al. Stem Cell Reports, 2018 Dec. 11, 11(6):1539-1550; Lees E A et al. J Vis Exp. 2019 May 12, (147);
a nephroid cell or another kidney-related cell as obtainable by the methods described in Vanslambrouck J M et al. J Am Soc Nephrol. 2019 October, 30(10):1811-1823;
a cardiomyocyte as obtainable by the method described in Huang C Y et al. J Mol Cell Cardiol. 2019 Oct. 23, 138:1-11;
a retinal cell, a retinal pigment epithelium cell as obtainable by the methods described in Ben M'Barek K et al. Biomaterials. 2019 Nov. 6:119603,
a mesenchymal stem cell as obtainable by the method described in Chen K H et al. Am J Transl Res. 2019 Sep. 15; 11(9):6232-6248).

In an embodiment of the method of the invention, where a differentiation step applies, the mammalian cell is an undifferentiated cell, such as stem cell, pluripotent cell or iPS cell, and is differentiated into a cell selected from the above list.

In an embodiment of the method of the invention, the implantable mammalian cell is a differentiated cell selected from the above list.

Non-limiting embodiments of the invention include:
1. Mammalian cell comprising at least a B2M/HLA-E gene wherein said mammalian cell comprises no other expressible B2M genes.
2. Mammalian cell according to embodiment 1 comprising a B2M/HLA-E*0101 gene and a B2M/HLA-E*0103 gene wherein said mammalian cell comprises no other expressible B2M genes.
3. Mammalian cell according to embodiment 1 comprising a B2M/HLA-E*0101 gene or a B2M/HLA-E*0103 gene.
4. The mammalian cell according to any of the previous embodiments, wherein said cell has knock-in of 4 or at least 4 HSV-TK genes at distinct and known locations.
5. The mammalian cell according to any of the previous embodiments, wherein said B2M/HLA-E*0101 and/or B2M/HLA-E*0103 genes have been knocked-in into the native B2M sequences of said mammalian cell.
6. Mammalian cell comprising B2M/HLA-E*0101 and B2M/HLA-E*0103 wherein said mammalian cell comprises no other expressible B2M genes.
7. Mammalian cell which has knock-ins of both B2M/HLA-E*0101 and B2M/HLA-E*0103 into an otherwise beta 2 microglobulin (B2M) deficient cell.
8. Mammalian cell according to any of embodiments 6-7, wherein said B2M/HLA-E*0101 and B2M/HLA-E*0103 have been knocked-in directly in the native B2M sequences of the cell used to make said B2M deficient cell.
9. Mammalian cell according to any of the preceding embodiments, wherein said mammalian cell has the HLA-A/B/C^{-/-} HLA-E⁺ cell surface phenotype, such as the HLA-A/B/C^{-/-} HLA-E*0103⁺ and/or HLA-A/B/C^{-/-} HLA-E*0101⁺ cell surface phenotype.
10. Mammalian cell according to any of the preceding embodiments, wherein said mammalian cell has the HLA-A/B/C^{-/-} HLA-E⁺ cell surface phenotype and comprises knock-ins of 4 HSV-TK genes at distinct and known locations.
11. Mammalian cell according to any of the preceding embodiments, wherein said mammalian cell has the HLA-A/B/C^{-/-} HLA-E*0101⁺ and HLA-E*0103⁺ cell surface phenotype.
12. Mammalian cell comprising B2M/HLA-E*0101 and B2M/HLA-E*0103 genes wherein said mammalian cell comprises no other expressible B2M genes, is CIITA deficient and has knock-ins of 4 HSV-TK genes at distinct and known locations.
13. Mammalian cell according to any of the preceding embodiments, wherein said mammalian cell is a universally transplantable cell.
14. Mammalian cell according to any of the preceding embodiments, wherein said mammalian cell is a stem cell or a pluripotent cell.

15. Mammalian cell according to any of the preceding embodiments, wherein said mammalian cell is selected from the group consisting of a neuron, a cardiomyocyte, retinal cell, a retinal pigment epithelium cell and a beta cell.
16. Mammalian cell according to embodiment 15, wherein said mammalian cell is a beta cell or a precursor thereof.
17. Mammalian cell according to any of the preceding embodiments, wherein said mammalian cell is selected from the group consisting of a mesenchymal stem cell, an embryonal stem cell, a neural stem cell.
18. Mammalian cell according to any one of preceding embodiments, wherein said B2M/HLA-E gene(s), such as B2M/HLA-E*0101 and/or B2M/HLA-E*0103 genes, each include a promoter or are knocked-in in loci that are under the control of a functional promoter or next to a promoter.
19. Mammalian cell according to any of the preceding embodiments, wherein the knock-in of said B2M/HLA-E genes is over the native B2M locus, utilizing (i.e. under the control of) the native B2M promoter.
20. Mammalian cell according to any of embodiments 1-19, wherein the knock-in of said B2M/HLA-E*0101 and/or B2M/HLA-E*0103 genes is over the native B2M loci and utilizes (i.e. is under the control of) a non-native B2M promoter.
21. Mammalian cell according to any of embodiments 1-19, wherein said B2M/HLA-E*0101 and/or B2M/HLA-E*0103 genes are knocked-in in loci other than the native B2M loci and that utilize an alternate promoter.
22. Mammalian cell according to any one of embodiments 1-21, wherein the desired HLA-E density is generated via bi-allelic HLA-E knock-ins.
23. Mammalian cell according to any one of the preceding embodiments, wherein no preferential loading of HLA-G signal sequence peptide is used.
24. Mammalian cell according to any one of the preceding embodiments, wherein the B2M/HLA-E gene does not comprise a pre-bound HLA-I leader peptide.
25. Mammalian cell according to any of the preceding embodiments, wherein said B2M/HLA-E*0101 gene encodes a B2M/HLA-E*0101 protein of the amino acid sequence SEQ ID NO:4 or a variant thereof having a total of 1-10 substitutions, deletions or additions.
26. Mammalian cell according to any of the preceding embodiments, wherein said B2M/HLA-E*0103 gene encodes a B2M/HLA-E*0103 protein of the amino acid sequence SEQ ID NO:5 or a variant thereof having a total of 1-10 substitutions, deletions or additions.
27. Mammalian cell according to any of the preceding embodiments, wherein said mammalian cell is HLA-II deficient, such as CIITA deficient.
28. Mammalian cell according to any of the preceding embodiments, which comprises knock-ins of 4 or at least 4 HSV-TK genes at distinct and known locations.
29. Mammalian cell according to embodiment 28 wherein said knock-ins of 4 HSV-TK genes are at locations separated by at least 10 Kbp, such as at least 100 Kbp, at least 1 Mbp or at least 20 Mbp.
30. Mammalian cell according to any of embodiments 28-29 wherein said knock-ins of 4 HSV-TK genes are at locations on 4 different chromosomes.
31. Mammalian cell according to any of embodiments 28-29 wherein said knock-ins of 4 HSV-TK genes are at locations on 3 different chromosomes.
32. Mammalian cell according to any of embodiments 28-29 wherein said knock-ins of 4 HSV-TK genes are at locations on 2 different chromosomes.
33. Mammalian cell according to embodiment 28, wherein said 4 HSV-TK genes are expressed to an extent where each of them alone would kill said mammalian cell upon exposure to ganciclovir.
34. Mammalian cell according to any of the preceding embodiments, wherein 2 or at least 2 HSV-TK genes are knocked-in at safe genomic harbour sites.
35. Mammalian cell according to any of the preceding embodiments, wherein one HSV-TK gene is knocked-in to eliminate a B2M allele.
36. Mammalian cell according to any of the preceding embodiments, wherein one HSV-TK gene is knocked-in to eliminate a CIITA allele.
37. Mammalian cell according to any of embodiments 4-36, wherein said 4 HSV-TK gene are knocked-in at safe harbour sites, such as AAVs1, hROSA, AAVS1, CLYBL or any combination thereof.
38. Mammalian cell according to any of the preceding embodiments, which mammalian cell is not a Natural Killer (NK) cell.
39. A method for making an implantable mammalian cell, comprising the steps of:
   providing a mammalian cell,
   knock-in of at least a B2M/HLA-E fusion gene, such as a B2M/HLA-E*0101 gene and/or a B2M/HLA-E*0103 gene, into said mammalian cell,
   inactivate the native B2M genes of said mammalian cell,
   optionally differentiate said mammalian cell,
      whereby said implantable mammalian cell is obtained.
40. Method for making an implantable mammalian cell, comprising the steps of:
   providing a mammalian cell,
   knock-in of at least a B2M/HLA-E fusion gene, such as a B2M/HLA-E*0101 gene and/or a B2M/HLA-E*0103 gene, into said mammalian cell,
   inactivate the native B2M genes of said mammalian cell,
   differentiate said mammalian cell, whereby said implantable mammalian cell is obtained.
41. Method for making an implantable mammalian cell, comprising the steps of:
   a) providing a mammalian cell,
   b) knock-in of a B2M/HLA-E gene into the B2M gene loci in said mammalian cell, whereby said implantable mammalian cell is obtained.
42. Method for making an implantable mammalian cell, comprising the steps of:
   a) providing a B2M deficient undifferentiated mammalian cell,
   b) knock-in of a B2M/HLA-E gene into said B2M deficient undifferentiated mammalian cell, and
   c) differentiating said undifferentiated cell into a functional differentiated cell, whereby said implantable mammalian cell is obtained.
43. Method for making an implantable mammalian cell, comprising the steps of:
   a) providing a B2M deficient mammalian cell,
   b) knock-in of a B2M/HLA-E gene, such as B2M/HLA-E*0101 and/or a B2M/HLA-E*0103 gene, into said B2M deficient mammalian cell, whereby said implantable mammalian cell is obtained.
44. Method according to any of embodiments 39-43 further comprising a step of: knock-in of at least 4 HSV-TK genes at distinct and known locations.
45. Method according to any of embodiments 39-44 wherein at least 2 HSV-TK genes are knocked-in at safe harbour loci.
46. Method according to any of embodiments 39-45 wherein 4 HSV-TK genes are knocked-in at safe harbour loci.
47. Method according to any of embodiments 39-46 further comprising a step of: inactivating the native HLA-II genes or the native CIITA genes of said mammalian cell.
48. Method according to any of embodiments 39-47, wherein said B2M/HLA-E genes, such as B2M/HLA-E*0101 and/or B2M/HLA-E*0103 genes, have been knocked-in directly over the native B2M sequences of the cell used to make said B2M deficient cell.
49. Method according to any of embodiments 39-48 wherein said B2M/HLA-E gene comprises a B2M/HLA-E*0101 gene and a B2M/HLA-E*0103 gene.
50. Method according to any of embodiments 39-49, wherein said mammalian cell as the cell surface phenotype of HLA-A/B/C^{-/-} HLA-E*0101⁺ HLA-E*0103⁺ cells.
51. Method according to any of embodiments 39-50, wherein said mammalian cell is a stem cell.
52. Method according to any of embodiments 39-51, wherein said mammalian cell or said transplantable mammalian cell is selected from a neuron, a cardiomyocyte, retinal cell, a retinal pigment epithelium cell, a mesenchymal stem cell and a beta cell.
53. Method according to any of embodiments 39-52 comprising the steps of:
   providing a mammalian stem cell or pluripotent cell,
   knock-in of at least a B2M/HLA-E*0101 gene and a B2M/HLA-E*0103 gene, into said mammalian cell,
   inactivating the native B2M genes of said mammalian cell,
   knock-in of at least 4 HSV-TK genes at distinct and known locations,
   differentiating said mammalian cell,
   whereby said implantable mammalian cell is obtained.
54. Method according to any of embodiments 39-53 wherein, in the differentiation step, said mammalian cell is differentiated into a beta cell, an INS+ and NKX6.1+ double positive cell or a C-peptide+/NKX6.1+ double positive cells, an insulin producing cell, an in vitro derived beta-like cell, a pancreatic endocrine cell or an endocrine cell, an endocrine progenitor cell or a NGN3+/NKX2.2+ double positive cell, a neural cell, such as a neuron, an interneuron cell, an oligodendrocyte, an astrocyte, a dopaminergic cell, an exosome cell, such as ESCs or NSCs, or an exosome cell derived from a ESC or NSC, an immune cell, such as a T cell, a NK cell, a macrophage, a dendritic cell, a hepatocyte, a stellate cell, a fibroblast, a keratinocyte or a hair cell, an inner ear cell, an intestinal cell or organoid cell, a nephroid cell or another kidney-related cell, a cardiomyocyte, a retinal cell, a retinal pigment epithelium cell, a mesenchymal stem cell.
55. Method according to any of embodiments 39-54 wherein said implantable mammalian cell is selected from a beta cell, an INS+ and NKX6.1+ double positive cell or a C-peptide+/NKX6.1+ double positive cells, an insulin producing cell, an in vitro derived beta-like cell, a pancreatic endocrine cell or an endocrine cell, an endocrine progenitor cell or a NGN3+/NKX2.2+ double positive cell, a neural cell, such as a neuron, an interneuron cell, an oligodendrocyte, an astrocyte, a dopaminergic cell, an exosome cell, such as ESCs or NSCs, or an exosome cell derived from a ESC or NSC, an immune cell, such as a T cell, a NK cell, a macrophage, a dendritic cell, a hepatocyte, a stellate cell, a fibroblast, a keratinocyte or a hair cell, an inner ear cell, an intestinal cell or organoid cell, a nephroid cell or another kidney-related cell, a cardiomyocyte, a retinal cell, a retinal pigment epithelium cell, a mesenchymal stem cell.
56. Method according to any of embodiments 39-55, wherein knocked-in B2M/HLA-E genes, such as B2M/HLA-E*0101 gene and/or B2M/HLA-E*0103 gene, each include a promoter or wherein said B2M/HLA-E genes are knocked-in next to a promoter or in loci under the control of functional promoter(s).
57. Method according to any of embodiments 39-56, wherein knock-in of said B2M/HLA-E*0101 gene and B2M/HLA-E*0103 gene is over the native B2M locus, utilizing the native B2M promoter.
58. Method according to any of embodiments 39-57, wherein knock-in of said B2M/HLA-E*0101 gene and B2M/HLA-E*0103 gene is over the native B2M locus, utilizing an alternate non-native B2M promoter.
59. Method according to any of embodiments 39-58, wherein knock-in of both B2M/HLA-E*0101 gene and B2M/HLA-E*0103 gene are in loci other than the native B2M loci and utilize or are under the control of an alternate promoter.
60. Method according to any of embodiments 39-59, wherein said B2M/HLA-E*0101 gene encodes a B2M/HLA-E*0101 protein of amino acid sequence SEQ ID NO:4 or a variant thereof having a total of 1-10 substitutions, deletions or additions.
61. Method according to any of embodiments 39-60, wherein said B2M/HLA-E*0103 gene encoded a B2M/HLA-E*0103 protein of amino acid sequence SEQ ID NO:5 or a variant thereof having a total of 1-10 substitutions, deletions or additions.
62. Method according to any one of embodiments 39-61, wherein the desired HLA-E density is generated via bi-allelic knock-ins.
63. Method according to any one of embodiments 39-62, wherein no preferential loading of HLA-G signal sequence peptide is used.
64. Method according to any one of embodiments 39-63, wherein said mammalian cell is CIITA deficient.
65. Method according to any one of embodiments 39-64, comprising a step of inactivating the expression of functional HLA-II proteins.
66. Method according to embodiment 65 comprising a step of inactivating the CIITA gene.
67. Method according to any of embodiments 41-66, which further comprises the step: c) knock-ins of 4 HSV-TK genes at distinct and known locations.
68. Method according to any of embodiments 44-67, wherein said knock-ins of 4 HSV-TK genes are at locations separated by at least 10 Kbp, such as at least 100 Kbp, at least 1 Mbp or at least 20 Mbp.
69. Method according to any of embodiments 44-68 wherein said knock-ins of 4 HSV-TK genes are at locations on 4 different chromosomes.
70. Method according to any of embodiments 44-69 wherein said knock-ins of 4 HSV-TK genes are at locations on 2 different chromosomes.
71. Method according to any of embodiments 44-70, wherein the HSV-TK proteins expressed by only one of said 4 HSV-TK genes are sufficient to kill said mammalian cell upon exposure to ganciclovir.
72. Method according to any of embodiments 44-71, wherein 2 or at least 2 HSV-TK genes are knocked-in at safe genomic harbour sites.
73. Method according to any of embodiments 44-72, wherein one HSV-TK gene is knocked-in to eliminate a B2M allele.
74. Method according to any of embodiments 44-73, wherein one HSV-TK gene is knocked-in to eliminate a CIITA allele.
75. Method according to any one of embodiments 39-74, wherein said knock-ins and/or gene inactivation(s) is/are conducted using a gene editing technology selected from Zinc finger nucleases (ZFNs), CRISPR, TALEN or adenoviral recombination.
76. Method according to any one of embodiments 39-75 wherein said B2M deficient mammalian cell is a stem cell which has been modified by knocking out both alleles of native B2M.
77. Use of a mammalian cell according to any one of embodiments 1-38 for the prevention, treatment or cure of a chronic disease.
78. Use according to embodiment 77, wherein said chronic disease is selected from the group consisting of diabetes, type 1 diabetes, type 2 diabetes, dry macular degeneration, retinitis pigmentosa, neurological disease, Parkinson's disease, heart disease, chronic heart failure and chronic kidney disease.
79. A mammalian cell according to any one of embodiments 1-38 wherein one HSV-TK gene is knocked-in to eliminate a B2M allele and another HSV-TK gene is knocked-in to eliminate a CIITA allele.
80. Method for making an implantable mammalian cell, comprising the steps of:
   providing a B2M deficient and CIITA deficient mammalian cell,
   knock-in of a B2M/HLA-E gene, such as one of or both a B2M/HLA-E*0101 and a B2M/HLA-E*0103 gene, into said B2M and CIITA deficient mammalian cell,
   knock-ins of 4 HSV-TK genes at distinct and known locations, whereby said implantable mammalian cell is obtained.
81. Method of embodiment 80, wherein said implantable mammalian cell has a of HLA-A/B/C−/− HLA-E cell surface phenotype.
82. Method of embodiment 80-81, wherein said implantable mammalian cell has the cell surface phenotype of HLA-A/B/C−/− HLA-E*0101+ HLA-E*0103+ cells.
83. Method according to any of embodiments 80-82, wherein said mammalian cell is a stem cell, a pluripotent cell or an iPS cell.
84. Method according to any of embodiments 80-83, wherein said mammalian cell is selected from a neuron, a cardiomyocyte, a retinal cell, a retinal pigment epithelium cell, a mesenchymal stem cell and a beta cell.
85. Method according to any of embodiments 80-84, comprising a step of differentiating said mammalian cell.
86. Method according to embodiment 85, wherein, in the differentiation step, said mammalian cell is differentiated into a beta cell, an INS+ and NKX6.1+ double positive cell or a C-peptide+/NKX6.1+ double positive cells, an insulin producing cell, an in vitro derived beta-like cell, a pancreatic endocrine cell or an endocrine cell, an endocrine progenitor cell or a NGN3+/NKX2.2+ double positive cell, a neural cell, such as a neuron, an interneuron cell, an oligodendrocyte, an astrocyte, a dopaminergic cell, an exosome cell, an immune cell, such as a T cell, a NK cell, a macrophage, a dendritic cell, a hepatocyte, a stellate cell, a fibroblast, a keratinocyte or a hair cell, an inner ear cell, an intestinal cell or organoid cell, a nephroid cell or another kidney-related cell, a cardiomyocyte, a retinal cell, a retinal pigment epithelium cell, a mesenchymal stem cell.
87. Method according to any of embodiments 80-84, wherein said knock-ins of 4 HSV-TK genes are at locations on 4 different chromosomes.
88. Method according to any one of embodiments 80-85, wherein 2 HSV-TK genes are knock-in at safe genomic harbour sites.

### EXAMPLES

### Example 1—Ganciclovir Assay

Description

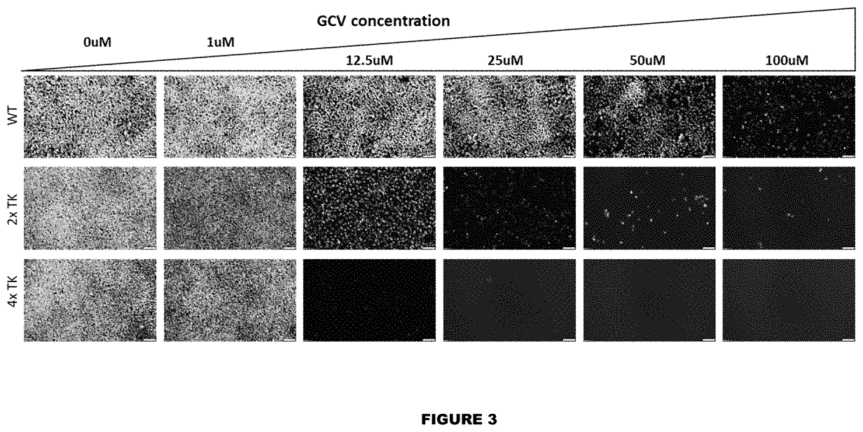

An undifferentiated parental hESC (Human Embryonic Stem Cell) cell line (WT), a hESC cell line with 2 copies of the HSV-TK gene (2×HSV-TK), and a hESC cell line with 4 copies of the HSV-TK gene (4×HSV-TK) were plated on hFN (human fibronectin coating). The cells were seeded at 60.000 cells/well in 24 wells format dishes and cultured overnight in DEF-CS media. The cells were then cultured in DEF-CS media containing ganciclovir (GCV) at 5 different concentrations for 7 days: 0, 1, 12.5, 25, 50 or 100 □M. DEF-CS media containing ganciclovir was changed every day. The cells were passaged 1:2 in DEF-CS with ganciclovir, when they reached 90% confluency. After 7 days in culture the cells were stained with DAPI and images were captured. The result images are shown in FIG. 3.

### CONCLUSION

Cells having four copies of HSV-TK at distinct sites in the genome are more sensitive toward ganciclovir, than cells only having two copies of HSV-TK at distinct sites in the genome.

### Example 2—Immune Safe Cells Generation Protocol

Human embryonic stem cells (SA121) are electroporated with a total of 500 ng TALEN® mRNA pair (ThermoFisher®, forward target sequence:

CTGTCCCCTCCACCCCAC, reverse target sequence:

TTCTGTCACCAATCCTGT) against AAVS1 and 500 ng donor plasmid containing 300 bp homology arms flanking the TALEN® cut site in AAVS1, an HSV-TK cassette followed by a mCherry selection cassette. The cells are cultured for a week and the mCherry positive cells are bulk sorted using a FACS cell sorter. The cells are cultured for an additional week before they are electroporated with a total of 500 ng TALEN® mRNA pair (ThermoFisher®, forward target sequence:

CTCAAGTAGGTCTCTTTC, reverse target sequence:

GAAAGTCTTCTCCTCCAA) against CLYBL and 500 ng donor plasmid containing 300 bp homology arms flanking the TALEN® cut site in CLYBL, a HSV-TK cassette followed by a eGFP selection cassette. Cells are cultured for one week and the mCherry/eGFP double positive cells are bulk sorted using a FACS cell sorter. The cells are cultured for an additional week before they are electroporated with 100 ng Cre recombinase mRNA to excise the selection cassettes. The mCherry/eGFP double negative cells are single cell sorted into a 96 well plate using a FACS cell sorter and cultured in for two to four weeks. The cell clones are screened for targeted bi-allelic integration using PCR.

A clone containing four HSV-TK copies from the protocol above is electroporated with a total of 200 ng TALEN® mRNA pair (ThermoFisher®, forward target sequence: TCTCGCTCCGTGGCCTT, reverse target sequence: AGCCTCCAGGCCAGAAAG) against B2M and 200 ng donor plasmid containing 300 bp homology arms flanking the TALEN® cut site in B2M, a B2M-HLAIE01:01 fusion cassette followed by a mCherry selection cassette and 200 ng donor plasmid containing 300 bp homology arms flanking the TALEN® cut site in B2M, a B2M-HLAIE01:03 fusion cassette followed by a eGFP selection cassette. Cells are cultured for one week and the mCherry/eGFP double positive cells are bulk sorted using a FACS cell sorter. The cells are cultured for an additional week before they are electroporated with 100 ng Cre recombinase mRNA to excise the selection cassettes. The mCherry/eGFP double negative cells are single cell sorted into a 96 well plate using a FACS cell sorter and cultured in for two to four weeks. The cell clones are screened for targeted mono-allelic integration using PCR

All the electroporation's are done using the 10 uL Neon transfection kit according the manufactures instructions (ThermoFisher®#MPK1025, Puls voltage 1100V, Pulse width 20, Pulse no 2, 4e5 cells).

Cells are cultured in DEF-CS according to manufacturer's instructions (Takare®#Y30017).

1. A mammalian cell comprising a B2M/HLA-E gene wherein said mammalian cell comprises no other expressible B2M genes and has knock-ins of at least 4 HSV-TK genes at distinct and known locations.

2. The mammalian cell according to claim 1, wherein said mammalian cell comprises B2M/HLA-E*0101 and B2M/HLA-E*0103 genes.

3. The mammalian cell according to claim 1, wherein said mammalian cell is a stem cell.

4. The mammalian cell according to claim 2, wherein said mammalian cell is a stem cell.

5. The mammalian cell according to claim 1, wherein said mammalian cell is selected from the group consisting of a neural cell, a neuron, an interneuron cell, an oligodendrocyte, an astrocyte, a dopaminergic cell, an exosome cell, a cardiomyocyte, a retinal cell, a retinal pigment epithelium cell, a mesenchymal stem cell, a beta cell, a INS+ and NKX6.1+ double positive cell, a C-peptide+ and NKX6.1+ double positive cell, an insulin producing cell, an in vitro derived beta-like cell, a pancreatic endocrine cell, an endocrine cell, an immune cell, a T cell, a NK cell, a macrophage, a dendritic cell, an hepatocyte, a stellate cell, a fibroblast, a keratinocyte, a hair cell, an inner ear cell, an intestinal cell or organoid cell, a nephroid cell and a kidney-related cell.

6. The mammalian cell according to claim 2, wherein said mammalian cell is selected from the group consisting of a neural cell, a neuron, an interneuron cell, an oligodendrocyte, an astrocyte, a dopaminergic cell, an exosome cell, a cardiomyocyte, a retinal cell, a retinal pigment epithelium cell, a mesenchymal stem cell, a beta cell, a INS+ and NKX6.1+ double positive cell, a C-peptide+ and NKX6.1+ double positive cell, an insulin producing cell, an in vitro derived beta-like cell, a pancreatic endocrine cell, an endocrine cell, an immune cell, a T cell, a NK cell, a macrophage, a dendritic cell, an hepatocyte, a stellate cell, a fibroblast, a keratinocyte, a hair cell, an inner ear cell, an intestinal cell or organoid cell, a nephroid cell and a kidney-related cell.

7. The mammalian cell according to claim 1, wherein said mammalian cell is HLA-II deficient.

8. The mammalian cell according to claim 1, wherein said mammalian cell is CIITA deficient.

9. The mammalian cell according to claim 2, wherein said mammalian cell is HLA-II deficient.

10. The mammalian cell according to claim 2, wherein said mammalian cell is CIITA deficient.

11. The mammalian cell according to claim 6, wherein said mammalian cell is CIITA deficient.

12. The mammalian cell according to claim 1, wherein at least 2 HSV-TK genes are knock-in at safe genomic harbour sites.

13. The mammalian cell according to claim 2, wherein at least 2 HSV-TK genes are knock-in at safe genomic harbour sites.

14. The mammalian cell according to claim 8, wherein one HSV-TK gene is knocked-in at a safe harbour site and another HSV-TK gene is knocked-in to eliminate a CIITA allele.

15. The mammalian cell according to claim 10, wherein one HSV-TK gene is knocked-in at a safe harbour site and another HSV-TK gene is knocked-in to eliminate a CIITA allele.

16. A method for making an implantable mammalian cell, comprising the steps of:
providing a mammalian cell,
knock-in of at least a B2M/HLA-E gene into said mammalian cell,
inactivating the native B2M genes of said mammalian cell,
knock-in of at least 4 HSV-TK genes at distinct and known locations,
optionally differentiating said mammalian cell,
whereby said implantable mammalian cell is obtained.

17. The method according to claim 16, wherein said implantable mammalian cell has the cell surface phenotype of HLA-A/B/C^{-/-} HLA-E*0101⁺ HLA-E*0103⁺ cells.

18. The method according to claim 16, wherein said mammalian cell is selected from the group consisting of a stem cell, a pluripotent cell or an iPS cell, an endocrine progenitor cell and a NGN3+/NKX2.2+ double positive cell

19. The method according to claim 16, wherein said implantable mammalian cell is selected from the group consisting of a neural cell, a neuron, an interneuron cell, an oligodendrocyte, an astrocyte, a dopaminergic cell, an exosome cell, a cardiomyocyte, a retinal cell, a retinal pigment epithelium cell, a mesenchymal stem cell, a beta cell, a INS+ and NKX6.1+ double positive cell, a C-peptide+ and NKX6.1+ double positive cell, an insulin producing cell, an in vitro derived beta-like cell, a pancreatic endocrine cell, an endocrine cell, an immune cell, a T cell, a NK cell, a macrophage, a dendritic cell, an hepatocyte, a stellate cell, a fibroblast, a keratinocyte, a hair cell, an inner ear cell, an intestinal cell or organoid cell, a nephroid cell and a kidney-related cell.

20. The method according to claim 16, wherein said knock-ins of 4 HSV-TK genes are at locations on 2 different chromosomes.

21. The method according to claim 16, wherein at least 2 HSV-TK genes are knocked-in at safe genomic harbour sites.

22. The method according to claim 17, wherein said mammalian cell is selected from the group consisting of a stem cell, a pluripotent cell or an iPS cell, an endocrine progenitor cell and a NGN3+/NKX2.2+ double positive cell.

23. The method according to claim 17, wherein said implantable mammalian cell is selected from the group consisting of a neural cell, a neuron, an interneuron cell, an oligodendrocyte, an astrocyte, a dopaminergic cell, an exosome cell, a cardiomyocyte, a retinal cell, a retinal pigment epithelium cell, a mesenchymal stem cell, a beta cell, a INS+ and NKX6.1+ double positive cell, a C-peptide+ and NKX6.1+ double positive cell, an insulin producing cell, an in vitro derived beta-like cell, a pancreatic endocrine cell, an endocrine cell, an immune cell, a T cell, a NK cell, a macrophage, a dendritic cell, an hepatocyte, a stellate cell, a fibroblast, a keratinocyte, a hair cell, an inner ear cell, an intestinal cell or organoid cell, a nephroid cell and a kidney-related cell.

24. The method according to claim 17, wherein said knock-ins of 4 HSV-TK genes are at locations on 2 different chromosomes.

25. The method according to claim 17, wherein at least 2 HSV-TK genes are knocked-in at safe genomic harbour sites.

26. A method of treating a chronic disease comprising administering a mammalian cell according to claim 1 to a subject in need thereof, wherein said disease is selected from the group consisting of diabetes, type 1 diabetes, type 2 diabetes, dry macular degeneration, retinitis pigmentosa, neurological disease, Parkinson's disease, heart disease, chronic heart failure and chronic kidney disease.

27. A method of treating a chronic disease comprising administering a mammalian cell according to claim 2 to a subject in need thereof, wherein said disease is selected from the group consisting of diabetes, type 1 diabetes, type 2 diabetes, dry macular degeneration, retinitis pigmentosa, neurological disease, Parkinson's disease, heart disease, chronic heart failure and chronic kidney disease.

28. A method of treating a chronic disease comprising administering a mammalian cell according to claim 10 to a subject in need thereof, wherein said disease is selected from the group consisting of diabetes, type 1 diabetes, type 2 diabetes, dry macular degeneration, retinitis pigmentosa, neurological disease, Parkinson's disease, heart disease, chronic heart failure and chronic kidney disease.
